(19) United States
(12) Patent Application Publication
(10) Pub. No.: US 2008/0004597 A1
(43) Pub. Date: Jan. 3, 2008

(54) Methods and devices for endocardiac access

(76) Inventors: Omar M. Lattouf, Atlanta, GA (US); Robert Michael Webster, Orlando, FL (US); Sarah Webster, Orlando, FL (US); Sameer Shums, Lilbuen, GA (US); Carribeth Ramey, Suwanee, GA (US); Amin Rahme, Atlanta, GA (US)

Correspondence Address: LAW OFFICE OF COLLEN A. BEARD, LLC, P. O. BOX 1064, DECATUR, GA, 30031-1064, US

(21) Appl. No.: 11/784,385

(22) Filed: Apr. 6, 2007

Related U.S. Application Data
(63) Continuation-in-part of application No. 10/313,198, filed on Dec. 6, 2002.
Application No. 10/313,198 is a continuation-in-part of application No. 10/295,390, filed on Nov. 15, 2002, now Pat. No. 6,978,176.
(60) Provisional application No. 60/340,062, filed on Dec. 8, 2001.
(60) Provisional application No. 60/365,918, filed on Mar. 20, 2002.
(60) Provisional application No. 60/369,988, filed on Apr. 4, 2002.

(51) Int. Cl. A61M 31/00

(52) U.S. Cl. 604/511; 604/101.05; 604/174

(57) ABSTRACT

Methods and devices for performing endocardiac treatments using an instrument port placed in the heart wall and an instrument guide which has a steerable tip and which is inserted through the instrument port, allows passage of an instrument therethrough into a heart chamber, and steers the functional tip of the instrument to the desired location for treatment.

### CROSS REFERENCE TO RELATED APPLICATIONS

This application is a continuation-in-part of application Ser. No. 10/313,198, filed on Dec. 6, 2002 which is a continuation-in-part of application Ser. No. 10/295,390, filed on Nov. 15, 2002 which claims the priority of provisional application Ser. No. 60/340,062, filed Dec. 8, 2001, provisional application Ser. No. 60/365,918, filed Mar. 20, 2002, and provisional application Ser. No. 60/369,988, filed Apr. 4, 2002. The entire contents of these applications are incorporated herein by reference.

### BACKGROUND OF THE INVENTION

The invention is in the field of cardiac health, more specifically in the field of minimally invasive methods for cardiac treatment procedures. In particular, the invention is directed to devices that facilitate access to and treatments on the interior of the heart.

Medical procedures on the heart can be performed inside the heart (endocardial) and on the outside of the heart (epicardial). Endocardial procedures require access to the interior of the heart, which can be accomplished percutaneously through the vasculature or directly, through the patient's chest and heart wall.

For percutaneous access, a catheter is typically inserted at the femoral or carotid artery and threaded into the heart via the vasculature. Travel of the catheter is monitored using a fluoroscope. Percutaneous treatment has several issues that make it less than desirable. For one thing, the catheters and tools that are used for percutaneous cardiac procedures are limited in size because they must be threaded through the vasculature into the heart. Where a guide catheter is used, only tools that are smaller than the catheter can be threaded through the catheter to the site of use. In cases where more than one type of tool is used, each tool must be threaded separately, adding to the length of the process.

Maneuverability of a catheter which is threaded such a long distance is limited, which means that it is difficult and sometimes impossible to locate the catheter tip exactly at the cardiac tissue where treatment is needed. This also adds to the total length of the procedure. Another issue with percutaneous access can be various vascular complications such as bleeding, dissection, and rupture of a blood vessel. Moreover, some areas of the heart are difficult to access percutaneously.

For direct access to the interior of the heart, physicians have traditionally used “open heart” surgical procedures. This involves a gross thoracotomy, usually in the form of a median sternotomy, to gain access to the thoracic cavity. A saw or other cutting instrument is used to cut the sternum longitudinally, allowing the rib cage to be spread apart. A large opening into the thoracic cavity is thus created, through which the surgeon can directly visualize and operate upon the heart. Of course, such an invasive procedure has consequences, such as typically an extended hospital stay and an increased risk of complications and pain.

Once the surgeon has accessed the thoracic cavity, and the exterior of the heart, he must gain access to the interior of the heart for endocardiac procedures. Opening up the heart surgically can only be done after placing the heart under cardioplegic arrest and maintaining circulation using cardiopulmonary bypass. Stopping the heart invites serious complications.

To avoid cardiac bypass, the surgeon must have a way to penetrate the heart wall with an instrument without losing a tremendous amount of blood. A hemostatic seal must be created around the instrument passed through the wall. One way to create a hemostatic seal is by using a purse-string suture around the instrument inserted through the heart wall. However, purse-string sutures are not always effective and do not easily allow the insertion of more than one instrument.

From the above discussion it is apparent that there is a need for devices and methods to access the inside of the heart other than percutaneously and directly via open heart surgery. There is a need for methods and devices to access the interior of the heart non-invasively. There is further a need for devices that allow instruments that have already been developed for percutaneous use to be used in non-invasive endocardiac procedures.

Accordingly, to avoid the disadvantages of both open heart surgery and percutaneous access, the present invention provides a method for minimally invasive access to the interior of the heart. An area of the heart that is preferably accessed is the apical area of the heart, which is the rounded inferior extremity of the heart formed by the left and right ventricles. In normal healthy humans it generally lies beneath the fifth left intercostal space from the mid-sternal line.

Access to the interior of the heart via the apex (trans-apica1 access) is taught in U.S. Pat. No. 6,978,176 to Lattouf This patent is primarily directed to mitral valve repair but the method taught therein is also described as being useful for other procedures such as ablation.

U.S. Pat. No. 6,629,534 to St. Goar et al. teaches another method for mitral valve repair using percutaneous access and instruments. The instruments are advanced to the mitral valve through the vasculature and are thus very flexible and small.

Many other medical procedures are accomplished via percutaneous access, also requiring instruments that are very flexible, and long and small enough in diameter to fit through the vasculature. These procedures could be accomplished more effectively and safely using the devices and methods of the present invention, either with the instruments that are already available for percutaneous access or with newly designed instruments.

An exemplary list of medical procedures that are typically done via percutaneous access that could alternatively be accomplished using the devices and method of the present invention are mitral valve repair, aortic valve repair, ablation, and placement of sensors.

### SUMMARY OF THE INVENTION

The present invention is directed to methods and devices for performing endocardiac treatments. The methods rely upon access to the interior of the heart through the heart wall.

The devices are an instrument port and an instrument guide, which can be used in combination or separately. The instrument port is placed in the heart wall and allows passage of the instrument guide or an instrument therethrough into a heart chamber. The port is anchored by a sealing device which also serve to reduce blood loss from the heart. The instrument guide can be used with a variety of instruments to guide the instrument into the area of the heart where the procedure is to be carried out and to steer the instrument functional head to the heart tissue to be treated.

The instrument guide may be introduced into the heart interior through the instrument port or it may be inserted through the heart wall through means known in the art, such as by using a puncture and purse string suture. The instrument port may be used in conjunction with the instrument guide and it can also be used directly with any number of other instruments.

In one aspect, the instrument guide is designed to receive an instrument that is designed for percutaneous access. These instruments are too flexible to be used in a “direct heart” procedure but can be used when inserted through the instrument guide of the invention which provides stability to the catheter.

The instrument guide optionally includes a hemostatic valve to prevent exegesis of blood and optionally includes steering means for positioning the tip of the guide (and any instrument carried thereby) at a desired location.

The invention will become more apparent from the following detailed description and accompanying exemplary drawings.

### BRIEF DESCRIPTION OF THE DRAWINGS

FIG. 1 is a perspective view of a patient's chest, partially illustrating the patient's heart with part of the heart wall removed to expose the left ventricular and atrium chambers and showing the devices of the invention in position.

FIG. 2 is a perspective view of the devices of the devices of the invention as positioned in the left ventricular apex of a heart wall.

FIG. 3 shows one embodiment of an instrument port of the invention.

FIG. 4 shows another embodiment of an instrument port of the invention.

FIG. 5 shows another embodiment of an instrument port of the invention.

FIG. 6 shows another embodiment of an instrument port of the invention.

FIG. 7 shows another embodiment of an instrument port of the invention.

FIG. 8 illustrates the instrument guide of the invention in greater detail.

### DETAILED DESCRIPTION OF THE INVENTION

The devices are an instrument port and an instrument guide, which can be used in combination or separately. The devices allow a physician to gain access to the interior of the heart, in a minimally invasive manner, so that he or she can perform a medical procedure therein. The instrument port is designed to be temporarily implanted through the heart wall and designed so that the instrument guide can pass through the port and into a heart chamber. The instrument guide is designed to be inserted through the instrument port and provide guidance to an instrument inserted through the guide into a chamber of the heart.

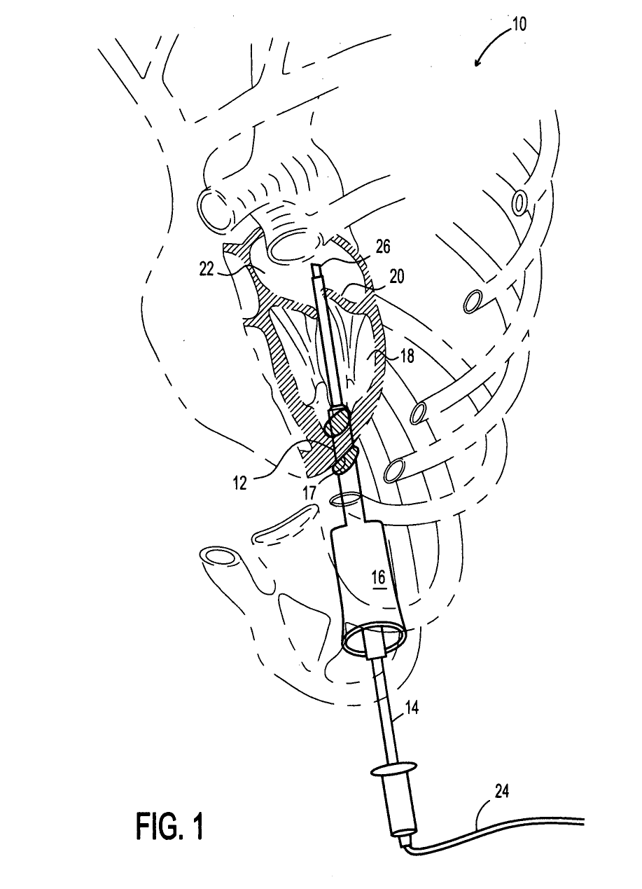

FIG. 1 illustrates the devices as used together as an instrument delivery system 10 in a human body to deliver an ablation catheter into the left atrium. The instrument port 12 is implanted at the apex 17 of the left ventricle. Instrument guide 14 is inserted through chest trocar 16, through the instrument port 12, into the left ventricle 18, past the mitral valve 20, and into the left atrium 22. An ablation catheter 24 is threaded through the instrument guide 14 so that its tip 26 is in the left atrium.

While the assembly is shown using the instrument port 12 in combination with the instrument guide 14 it should be understood that either component can be used without the other. The instrument port 12 can be used directly with an instrument, such as an ablation catheter. In this case the ablation catheter, for example, would desirably be one specially designed for so that it avoids the before mentioned issues of percutaneous catheters.

It should also be understood that although the system 10 is shown inserted through the apex of the left ventricle and positioned for use in the left atrium, it can be inserted through any area of the heart wall and used in any area of the heart. In addition, although the system 10 is shown for delivery of an ablation catheter it can be used with a wide variety of instruments and in a wide variety of procedures.

The instrument guide 14 could be inserted directly through the heart wall, as taught by the prior art, and a purse string suture used to prevent blood leakage. The instrument guide 14 could alternatively be used with another instrument port.

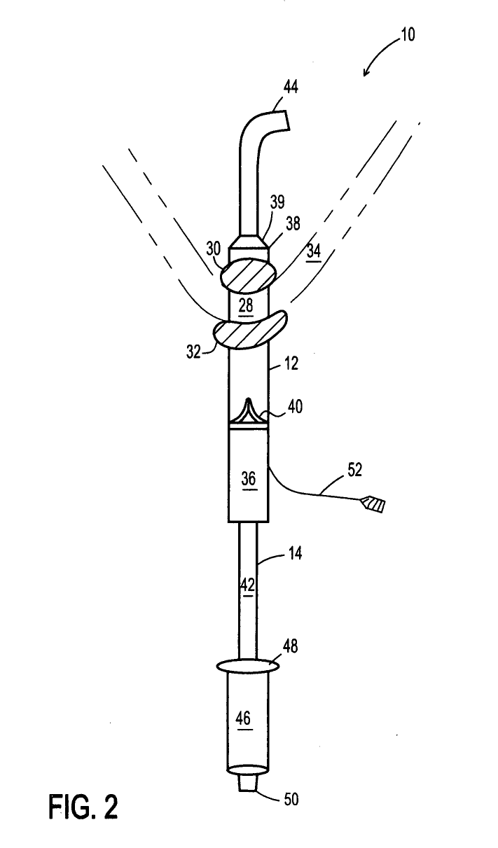

FIG. 2 illustrates the instrument delivery system 10 in greater detail with the instrument port 12 inserted through the heart wall 34. Instrument port 12 desirably has a cylindrical body with a heart wall portion 28 that generally is the width of the heart wall 34, and sealing device 30 and 32. In the embodiment shown the sealing device is two balloons, one on either side of the wall portion 28. The sealing device may however be a single balloon crimped in the middle, where the crimped part of the balloon is on the body wall portion 28 and a portion of the balloon extends from either side of the wall portion 28 and the heart wall 34 when the port is in place. In either case the sealing device may be a dog-bone shaped balloon. “Dog-bone shaped balloon” as used herein means a single balloon that is crimped so that it appears to be two balloons or two balloons arranged so that they have the profile of a dog bone. The sealing device serves to prevent blood from leaving the heart chamber and can be a variety of designs which serve that function. The sealing device may also serve to hold the port in place within the heart wall. In one embodiment, not illustrated, the sealing device of the port is a single balloon on the side of the port on the inside of the heart wall.

Various alternative designs for the instrument port are described below.

The instrument port desirably has a length from about 5 to 25 cm with a shaft portion 36 at its proximal end that is desirably about 1 mm to 15 cm in length. This shaft portion can be flared or otherwise differently shaped to allow easy insertion of the instrument guide 14 or other instrument therethrough. The opposite, distal, end of the instrument port 12 can be from about 0.5 to 5 cm in length. The distal tip 39 of the port, measuring about 0.5 to 1 cm in length, is desirably tapered and is radiopaque for visualization.

Wall portion 28 of the instrument port 12 is defined by the sealing device on either end, the balloons 30 and 32 as shown in FIG. 2. The width of wall portion 28 is desirably about the same as the thickness of the wall through which the port 12 is inserted. In most cases this will be from about 5 to 40 mm. The instrument port can have a wall portion of a set length or, in alternate embodiments, the instrument port has a variable length wall portion. Designs for instrument ports 12 having variable length wall portions are discussed below.

The outer diameter of the instrument port 12 is desirably from about 1 to 20 mm and the inner diameter is desirably about 1 to 15 mm. This allows passage of an instrument guide or instrument through the port of up to 15 mm (45 Fr). The port 12 includes a one way valve 40 in the inner lumen so that blood is prevented from exiting the heart but so that the instrument guide 14 can be inserted through the inner lumen. The valve is desirably a hemostatic valve, such as a duck-bill valve, and is desirably made of silicon although other types of valves and materials can be used.

The instrument port is desirably made of polyether block amides known as PEBAX® polymers or other plasticizer-free thermoplastic elastomers. The balloons can be made of standard material for such items such as polyurethane and can be up to about 2.5 cm in size when inflated. The balloons are filled via inflation port 52. The embodiment is shown with one inflation port for both balloons 30, 32 but they could alternatively be filed via separate inflation ports.

Instrument guide 14 has a body portion 42 with optionally but desirably a steerable tip 44. Handle 46 includes optional thumb knob 48 for steering control. A hemostatic valve 50 is shown at the distal end of handle 46. The optional hemostatic valve 50 prevents blood from exiting through the instrument guide 14 while allowing passage of instruments through the instrument guide lumen and can be located anywhere in the lumen of the instrument guide 14.

The instrument guide body 42 is desirably from about 8 to 18 inches in length, where the distal 4 inches is the steerable tip. The body 42 is desirably made of a stiff material such as PEBAX® or polystyrene for the non-steerable part of the body and a softer material such as polyurethane for the steerable portion. The body portion is desirably stiff enough to be pushable and maneuverable and the tip is desirably soft enough to be steerable as described below. The outer diameter of the instrument guide is preferably about 5 to 45 French.

One or more lumens (not shown) run the length of the instrument guide 14. At least one lumen is dedicated for receiving one or more instruments to be used for completing a medical procedure in the heart. This lumen should be large enough to accept an instrument ranging in diameter from about 2-30 Fr. Other lumens may be provided for steerability, visualization, balloon inflation, and any other capability that is needed.

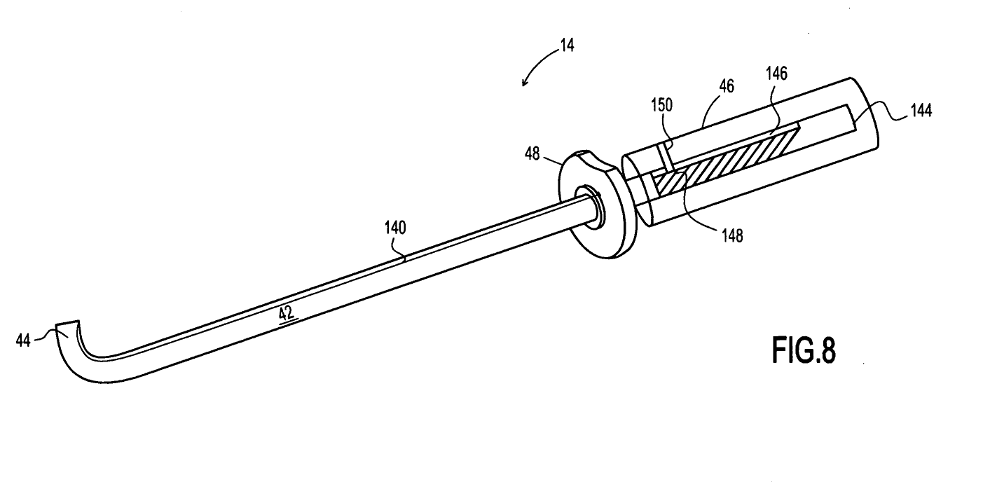

Steerable tip 44 can be controlled by various means. Desirably the tip can be rotated 360 degrees and bent at an angle up to 180 degrees. FIG. 8 illustrates one means of steering the distal tip 44. As shown in FIG. 8, the instrument guide 14 includes outer handle 46 and inner handle 144. The guide body 42 is formed as one piece with the inner handle 144, with the thumb knob 48 therebetween. Inner handle 144 includes a longitudinal slot 146 and a plurality of receiver grooves 148 extending from the longitudinal slot 146. The grooves 148 extend at an angle forward, towards the distal tip, from the longitudinal slot 146. Outer handle 46 has a detent 150 on the inside surface thereof which slides within the longitudinal slot 146 and mates with one of the receiver grooves 148.

A steering wire 140 is fastened to the distal tip 44 using adhesive or a swaged collar, for example. The other end of the steering wire is fastened to the outer handle 46. Pulling the outer handle 46 away from the tip causes the wire to tension and the distal tip 44 to bend. When the outer handle is slid away from the tip, the detent 150 slides in the longitudinal slot 146. When the distal tip 44 is bent to the desired angle, the outer handle is rotated, rotating the detent 150 within one of the receiver grooves 148 and locking the handle and thus the bend of the distal tip 44 in place.

To provide further locking ability, the detent can be modified with a spring mechanism to maintain tension and position.

After the distal tip 44 is bent, the instrument guide 14 can be rotated if the tip is not pointed in the correct direction by simply twisting the entire device. The instrument guide 14 will desirably rotate within the instrument port 12.

Other means for making a steerable tip are known in the art and can be used. For example, one method is to use a preformed bent tip and a stiffening wire that straightens the tip to the desired bend as it is pushed within the tip.

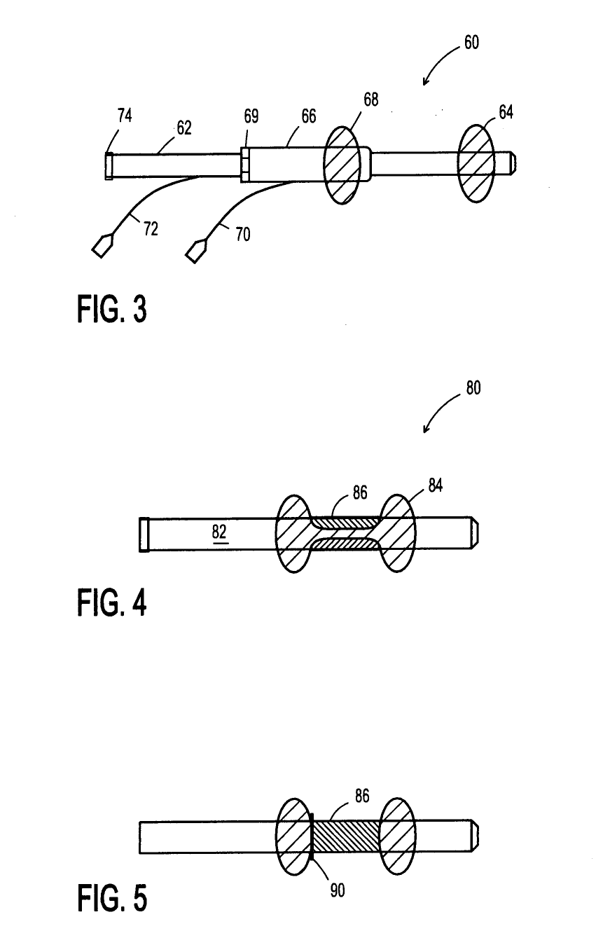

FIGS. 3-7 illustrate alternate embodiments of the instrument port. As discussed above, the length of the wall portion is desirably about the same as the thickness of the wall through which the port is inserted. The thickness of the heart wall varies from about 5 to 40 mm so an instrument port having a variable length wall portion would be useful.

In FIG. 3, the instrument port 60 is assembled from two cylindrical tube pieces assembled in a slidable coaxial relationship. An inner piece 62 includes a first, distal, balloon 64. An outer piece 66 includes a second, proximal, balloon 68. The pieces 62 and 66 are assembled in a coaxial sliding assembly so that the distance between the balloons 64 and 68 can be varied. A locking nut 69 on the proximal end of the second, outer piece 66 keeps the tubes 62 and 66 from sliding once they are in position. Inflation ports 70 and 72 are used to fill the balloons 68 and 64, respectively.

Rather than the internal one-way valve as shown in FIG. 1 above, this embodiment has a hemostatic valve 74 on the distal tip of the first inner piece 62. Either arrangement is possible for all embodiments described herein. Preferably both pieces 62 and 66 are long enough to extend out of the patient's chest so they can be easily manipulated.

FIG. 4 illustrates an instrument port 80 having a cylindrical body portion 82 and a single balloon 84. The balloon 84 is constrained with a spacer 86 of a certain length. The spacer 86 length approximates the heart wall thickness where the port 80 is to be installed. The spacer can be slid over one end of the port or may be made of a material that allows it to be spread open so that it can be placed on the port and then contracted once it is in place. The spacer 86 may optionally be crimped or glued in place or otherwise attached to the balloon. A similar port (not shown) has two balloons and uses a spacer to define a set distance between the balloons when they are inflated.

In the embodiment shown in FIG. 5, the spacer 86 includes a stop 90 on the distal end thereof, so that as the port is inserted into the heart wall it will only be inserted as far as the stop 90. A stop can be incorporated into any of the instrument ports described in this application.

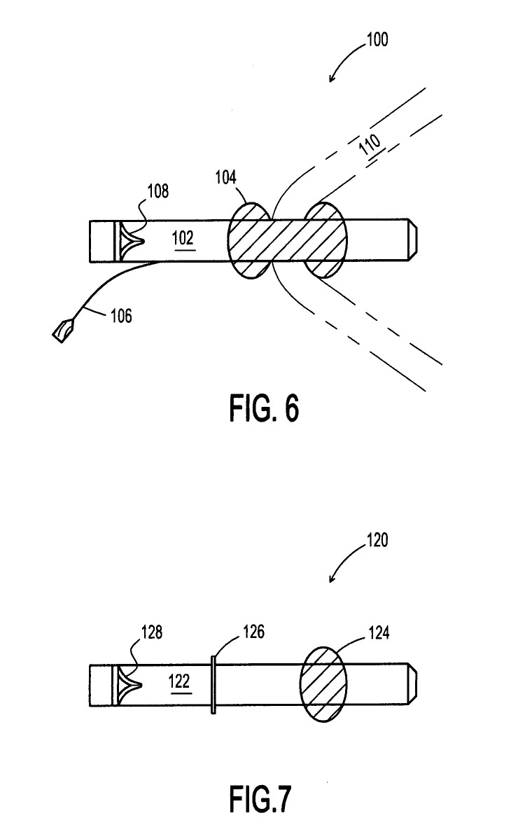

FIG. 6 illustrates an instrument port 100 having a cylindrical body portion 102 and a single balloon 104 inflated by inflation port 106. Duck-bill valve 108 is internal to the body portion 102. This port 100 forms a dog bone shape balloon when inserted into place in the heart wall 110 and inflated.

FIG. 7 shows an instrument port 120 having a cylindrical body 122 and a single balloon 124 designed to be placed inside the heart wall. A stop 126 is located on the body 122 a distance away from the balloon 124 that will approximate the thickness of the heart wall. The port further includes a valve 128.

The various components of the ports described here can be interchanged. For example, any of the ports can include a stop, to prevent the port from being inserted all the way through the heart wall. Any of the ports can include a spacer to define the space between the balloons, or between a balloon and a stop. Any of the ports can have a single balloon.

Any of the instrument ports described in this application can have one or more markers placed thereon so that they are visible by visualization means. For example, markers can be placed on either side of either or both balloons so that the physician can “see” where the port is in relation to the heart wall. Another way to promote visualization is using contrast agent in the balloon inflation media.

The procedure for using the devices generally includes first gaining access to the patient's chest cavity through a small opening made in the patient's chest, preferably though an intercostal space between two of the patient's ribs. Such accessing can be effected thorocoscopically through an intercostal space between the patient's ribs by minimally invasive procedures wherein a trocar or other suitable device is placed within the small opening made in the patient's chest.

To the extent required, the patient's deflated lung is moved out of the way, and then the pericardium on the patient's heart wall is removed to expose a region of the epicardium. The patient's heart wall is pierced at the exposed epicardial location to provide a passageway through the heart wall to a heart cavity such as the left ventricle. For the purposes of the discussion herein, the passageway is formed through a region of the heart wall at or near the apex of the patient's heart. A suitable piercing element includes a 14 gauge needle. A guide wire is advanced through the inner lumen of the needle into the heart chamber to the area of the heart to be treated. The penetrating needle may then be removed leaving the guide wire in place.

A sequence of progressively larger dilators can be inserted through the heart wall sequentially over the guidewire in predilation until the hole formed in the heart wall is large enough to accept the instrument port 12. The instrument port 12 (with the balloons deflated and properly folded) is then inserted over the last dilator. The dilator is removed and the balloons are inflated, holding the port in place and preventing or greatly reducing blood seepage from the heart.

Other methods of installing the instrument port 12 can be used. For example, a sheath can be placed over the last dilator, the dilator removed and then the port inserted into place through the sheath.

Once the instrument port 12 is in place, the instrument guide 14 is inserted through the instrument port 12, using the guidewire. After the instrument guide is in place, the guidewire is removed and the assembly is ready for use.

Various procedures can be performed using the endocardiac access system. For example, the system can be used in the mitral valve repair procedure discussed in U.S. Pat. No. 6,978,176 to Lattouf, Endocardial ablation can be performed, using, for example, percutaneous ablation catheters sold by various companies that utilize different energy sources such as radiofrequency, cryogenesis, ultrasound, microwave, radiation (beta source), or laser. For example, St. Jude Medical sells the Epicor technology that utilizes high intensity focused ultrasound (HIFU). Cryocath Inc. markets a circular cryocatheter called the Artic Circler. Cardima sells the Revelation Helix.

Once the procedure is complete, the instruments and instrument guide are removed, the port is removed and the heart wall opening is sutured. A plug can be inserted into the heart wall opening if desired.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A transcardiac instrument port for placement in a heart wall to allow passage into a chamber of the heart, comprising:
a cylindrical body portion defining a lumen and having an outer surface, a heart wall portion, and distal and proximal ends;
a valve associated with the lumen that allows passage of an instrument through the lumen but minimizes flow of blood out of the heart through the lumen; and
a sealing device on the body portion outer surface that minimizes blood flow from out of the heart around the outside of the port.

2. The instrument port of claim 1, wherein the sealing device is a dog-shaped balloon that can be inflated and that has a bulge on either side of the body portion wall portion when inflated.

3. The instrument port of claim 1, wherein the sealing device is two balloons, one on either side of the heart wall portion.

4. The instrument port of claim 1, wherein the sealing device is a single balloon.

5. The instrument port of claim 4, wherein the single balloon is crimped with a restraint having a length that approximates the width of the heart wall where the instrument port is to be inserted.

6. The instrument port of claim 1, wherein the cylindrical body portion comprises two cylindrical pieces in a slidable coaxial relationship.

7. The instrument port of claim 1, comprising a stop on the proximal side of the heart wall portion that stops movement of the port through the heart wall into the heart chamber.

8. An instrument guide for guiding an instrument to a desired location inside a heart and steering the tip of the instrument to a desired location inside the heart, comprising:
a handle;
a body portion defining a lumen to allow passage of an instrument therethrough and having a length from about 8 to 12 inches; and
a steerable tip that is positionable at a desired bend;
wherein the body portion is stiff relative to the steerable tip and is pushable with minimal bending.

9. The instrument guide of claim 8, wherein the steerable tip is rotatable 360° and bendable 180°.

10. The instrument guide of claim 8, wherein the steerable tip is bendable 90°.

11. An endocardiac access system for forming a passageway through a heart wall and guiding an instrument functional tip into position at a desired location in a heart chamber, comprising an instrument port for placement in the heart wall and an instrument guide for insertion through the instrument port, wherein:
the instrument port comprises a cylindrical body portion defining a lumen and having an outer surface, a heart wall portion, and distal and proximal ends;
a valve associated with the lumen that allows passage of an instrument through the lumen but minimizes flow of blood out of the heart through the lumen; and
a sealing device on the body portion outer surface that minimizes blood flow from out of the heart around the outside of the port; and wherein
the instrument guide comprises a body portion defining a lumen and a steerable tip and wherein an instrument can be inserted through the instrument guide lumen and the functional tip of the instrument guided to the desired location.

12. The endocardiac access system of claim 11, wherein the instrument port sealing device is a dog-shaped balloon that can be inflated and that has a bulge on either side of the body portion wall portion when inflated.

13. The endocardiac access system of claim 11, wherein the sealing device is two balloons, one on either side of the heart wall portion.

14. The endocardiac access system of claim 11, wherein the cylindrical body portion comprises two cylindrical pieces in a slidable coaxial relationship.

15. The endocardiac access system of claim 11, wherein the instrument guide body portion has a length from about 8 to 12 inches.

16. The endocardiac access system of claim 11, wherein the instrument steerable tip is rotatable 360° and bendable 180°.

17. The endocardiac access system of claim 11, wherein the instrument steerable tip is bendable 90°.

18. A minimally invasive method for forming a passageway for an instrument through a heart wall while minimizing blood flow out of the heart, comprising the steps,
piercing the heart wall to form a hole therethrough;
inserting an instrument port through the hole, wherein the instrument port comprises a cylindrical body portion defining a lumen and having an outer surface, a heart wall portion, and distal and proximal ends, a valve associated with the lumen that allows passage of an instrument through the lumen but minimizes flow of blood out of the heart through the lumen, and a sealing device on the body portion outer surface that minimizes blood flow from out of the heart around the outside of the port; and
inserting an instrument guide through the lumen of the instrument port, wherein the instrument guide comprises a body portion and a steerable flexible tip.

19. A method for using a percutaneous access catheter in a minimally invasive endocardiac procedure, comprising the steps,
piercing the heart wall to form a hole therethrough;
inserting an instrument port through the hole, wherein the instrument port comprises a cylindrical body portion defining a lumen and having an outer surface, a heart wall portion, and distal and proximal ends, a valve associated with the lumen that allows passage of an instrument through the lumen but minimizes flow of blood out of the heart through the lumen, and a sealing device on the body portion outer surface that minimizes blood flow from out of the heart around the outside of the port;
inserting an instrument guide through the lumen of the instrument port, wherein the instrument guide comprises a body portion and a steerable flexible tip; and
inserting the percutaneous access catheter through the instrument guide so that the distal tip of the percutaneous catheter extends from the distal tip of the instrument guide and can be guided to the desired treatment location by movement of the instrument guide steerable tip.

20. An assembly for using a percutaneous access catheter in a minimally invasive endocardiac procedure comprising:
a percutaneous access catheter having a functional tip;
an instrument port for placement in the heart wall and forming a passageway through the heart wall; and
an instrument guide for insertion through the instrument port and having a relatively stiff body portion and a steerable tip;
wherein the percutaneous access catheter can be inserted through the instrument guide and its functional tip steered to the desired location within the heart.
